(19) United States
(12) Patent Application Publication
(10) Pub. No.: US 2016/0316726 A1
(43) Pub. Date: Nov. 3, 2016

(54) FLOATING DEVICE FOR GROWING FISH AND/OR ALGAE

(71) Applicant: NEOTOP WATER SYSTEMS LTD., Tel Aviv (IL)

(72) Inventor: Zeev Birger, Ramat-HaSharon (IL)

(73) Assignee: NEOTOP WATER SYSTEMS LTD., Tel Aviv (IL)

(21) Appl. No.: 15/107,466

(22) PCT Filed: Dec. 23, 2014
(86) PCT No.: PCT/IL2014/051121

§ 371 Date: Jun. 23, 2016

Related U.S. Application Data
(60) Provisional application No. 61/920,786, filed on Dec. 26, 2013.

(51) Int. Cl. A01K 61/00; A01G 7/04; A01K 63/04; A01K 63/00; B63B 38/00; A01M 1/02; A01M 1/10; A01M 1/20; B65D 88/34; C12N 1/12; A01K 63/06; A01M 29/32

(52) U.S. Cl. CPC A01K 61/007; A01K 63/065; A01G 7/045; A01K 63/04; A01K 63/006; A01M 29/32; A01M 1/02; A01M 1/106; A01M 1/2094; A01M 1/2016; A01K 63/06; B65D 88/34; C12N 1/12; B63B 38/00

(57) ABSTRACT

A floating device for use on a water surface is disclosed. The floating device comprises: a generally spherical hollow body defined by an equator line, a top pole and a bottom pole. The floating device further comprises a non-flat floating member, being inside the hollow body and having an apex constituted to support the top pole when the floating device is placed on the water surface. The body of the floating device is formed with at least one opening at each of the top and the bottom poles, and with an arrangement of openings circumferentially distributed above and below the equator line.

### RELATED APPLICATION

This application claims the benefit of priority of U.S. Provisional Patent Application No. 61/920,786 filed Dec. 26, 2013, the contents of which are incorporated herein by reference in their entirety

### FIELD AND BACKGROUND OF THE INVENTION

The present invention, in some embodiments thereof, relates to algae and fish, more particularly, but not exclusively, to a method and floating device for growing fish and/or algae.

Covering liquid surfaces is a concern in many industries and public sectors. Several techniques have been proposed for such covering, from a spread of a thin layer of floating liquid, such as oil, on the surface to be covered, to the use of large solid films. Of particular relevance are individual floating devices that collectively form a barrier between a surface of liquid and the environment. One example is hollow spherical balls manufactured by Euro-Matic of Wilson, N.C. When deposited on the surface of the liquid, the balls arrange themselves into a cover. Also known are floatable spheroids having flat surfaces surrounding its equatorial plane systems (U.S. Pat. No. 3,998,204). The flat surfaces allow the spheroids to pack closer together on the fluid surface.

International Patent Publication No. WO2010/014879 teaches float members configured to float on and cover a fluid surface to modify transfer of evaporated fluid therethrough. Each float member is elongated and shaped like a disk with a cross-section having a rhombus shape. The sides of the float member are configured to interlock with adjacent members or have means for attracting adjacent float members to prevent gaps from opening.

International Patent Publication No. WO2011/161675 teaches a floating device which comprises a generally spherical hollow body having a first hemisphere and a second hemisphere connected to each other by a plurality of snap connectors. A floating member is disposed within an anterior of the body approximately at or near an equator line of the body, such that there is a gap between the floating member and the body.

Additional background art includes U.S. Pat. Nos. 248,796, 374,943, 2,553,798, 3,147,067, 3,462,040, 3,683,428, 3,687,329, 3,694,837, 3,872,522, 3,998,204, 3,938,338, 3,984,881, 3,984,882, 4,270,232, 4,022,187, 4,366,806, 4,458,668, 4,749,606 and 5,188,550, and Publication Nos. WO199812392, WO2006/010204, GB1008495, NL1002693 and AU2004100619.

### SUMMARY OF THE INVENTION

According to an aspect of some embodiments of the present invention there is provided a floating device for use on a water surface. The floating device comprises: a generally spherical hollow body defined by an equator line, a top pole and a bottom pole. The floating device further comprises a non-flat floating member, being inside the hollow body and having an apex constituted to support the top pole when the floating device is placed on the water surface. The body of the floating device is formed with at least one opening at each of the top and the bottom poles, and with an arrangement of openings circumferentially distributed above and below the equator line.

According to an aspect of some embodiments of the present invention there is provided a method of at least partially covering a liquid surface. The method comprises: placing a plurality of floating devices on the liquid surface, wherein each of at least a few of the plurality of floating devices is the floating device, as delineated above and optionally exemplified below.

According to some embodiments of the invention the method comprises on the liquid surface a linear floating element enclosing an area over the liquid surface, wherein the plurality of floating devices are placed to cover the enclosed area.

According to an aspect of some embodiments of the present invention there is provided a method of growing algae. The method comprises placing on a water surface a floating device, and allowing algae culture to grow therein. In various exemplary embodiments of the invention the floating device comprises a generally spherical hollow body defined by an equator line, a top pole and a bottom pole. The floating device also comprises a floating member inside the hollow body. The body is optionally and preferably formed with at least one opening at each of the top and the bottom poles, and with an arrangement of openings circumferentially distributed above and below the equator line.

According to some embodiments of the invention a shape of the opening at the top pole differs from a shape of the floating member such that when the floating member engages the opening at the top pole, sealing of the opening at the top pole by the floating member is prevented.

According to an aspect of some embodiments of the present invention there is provided a method of growing fish. The method comprises placing on a water surface a floating device, and allowing fish to enter the floating device. In various exemplary embodiments of the invention the floating device comprises a generally spherical hollow body defined by an equator line, a top pole and a bottom pole. The body is optionally and preferably formed with at least one opening at each of the top and the bottom poles, wherein the opening(s) is/are sizewise compatible with a size of the fish. The floating device optionally and preferably comprises a non-flat floating member, which is inside the hollow body and which has an apex constituted to support the top pole when the floating device is placed on the water surface. In various exemplary embodiments of the invention sealing of the opening at the top pole by the floating member is prevented.

According to an aspect of some embodiments of the present invention there is provided a method of trapping insects flying at a water surface. The method comprises placing on the water surface a floating device, and allowing the insects to enter the floating device. In various exemplary embodiments of the invention the floating device comprises a generally spherical hollow body defined by an equator line, a top pole and a bottom pole. The floating device optionally and preferably comprises a floating member inside the hollow body. The body is optionally and preferably formed with at least one opening at each of the top and the bottom poles, and with an arrangement of openings circumferentially distributed above and below the equator line.

According to some embodiments of the invention the floating member is generally shaped as a ring. According to some embodiments of the invention an inner diameter of the ring is selected to reduce evaporation.

According to some embodiments of the invention a largest dimension of the floating member is smaller than a radius of the hollow body by at least 5%.

According to some embodiments of the invention the floating member is generally shaped as a sphere.

According to some embodiments of the invention the floating member is generally shaped as a spheroid.

According to some embodiments of the invention the floating member has dimensions selected such that a fraction of its volume submerges under the water surface while an apex of the floating member supports the top pole to maintain the equator line generally at a level of the water surface. According to some embodiments of the invention the fraction is from about 0.03 to about 0.12.

According to some embodiments of the invention the floating device comprises flow redirectors at least partially surrounding each of the openings at the top and the bottom poles at an interior of the body.

According to some embodiments of the invention each of the flow redirectors is configured for establishing spiral flow about a respective pole when the pole is submerged under the water surface.

According to some embodiments of the invention each of the flow redirectors comprises a plurality of curved structures separated from each other by at least the top pole.

According to some embodiments of the invention at least one of the flow redirectors spirally winds about one of the poles over an inner surface of the body towards the equator line, wherein the floating member engages the spiral redirector when the device is placed on a liquid surface.

According to some embodiments of the invention the spiral redirector is non-continuous.

According to some embodiments of the invention the opening at the top pole has a diameter selected to reduce evaporation.

According to some embodiments of the invention an inner wall of the body has roughness features thereon.

According to some embodiments of the invention the roughness features are elongated and oriented generally along meridians of the body.

According to some embodiments of the invention the floating device comprises a pair of peripheral rims, mounted or formed on an external wall of the body above and below the equator line and generally parallel thereto.

According to some embodiments of the invention at least one of the peripheral rims is formed with roughness features.

According to some embodiments of the invention the roughness features of the at least one peripheral ring are at a side of the at least one peripheral ring which is opposite to the equator line.

According to some embodiments of the invention the floating device comprise a plurality of external wings, mounted or formed on an external wall of the body generally along meridians thereof.

According to some embodiments of the invention the water surface is a water surface of an outdoor water reservoir.

According to some embodiments of the invention the water surface is a water surface of an aquaculture pond.

According to some embodiments of the invention the water surface is a water surface of an aquarium.

According to some embodiments of the invention the body is transparent to visible light and the floating member has a dark color.

According to some embodiments of the invention the floating member comprises a thermally conductive material.

According to some embodiments of the invention the device comprises a light source positioned at an upper hemisphere of the body.

According to some embodiments of the invention the device comprises a solar power source configured for powering the light source.

According to some embodiments of the invention the device comprises a dissolvable substance placed at the inner side of a lower hemisphere of the device.

According to some embodiments of the invention the body comprises additives selected to selectively allow transmission of light having wavelength from about 550 nm to about 850 nm.

According to some embodiments of the invention the body is constructed to prevent birds from landing on the body when the body is placed on a liquid surface.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are schematic perspective views of a floating device, according to various exemplary embodiments of the present invention.

FIGS. 2A-C are schematic exploded views of a floating device, according to various exemplary embodiments of the present invention.

FIG. 2D is a schematic illustration showing nesting of a plurality of hemispheres, according to various exemplary embodiments of the present invention.

FIG. 3 is a cross-sectional illustration of a floating device, once placed on a water surface, in embodiments of the invention in which a spherical floating member is employed;

FIGS. 4A-C are schematic illustrations of a hemisphere of a floating device, according to some embodiments of the present invention;

FIGS. 4D and 4E are schematically illustrations showing embodiments of the invention in which one or more flow redirectors spirally wind about one of the poles of the floating device;

FIGS. 4F and 4G are schematically illustrations showing embodiments of the invention in which the shape of one or more of the openings at the poles of the floating device differs from the shape of a floating member of the floating device;

FIG. 5 is a schematic illustration of a floating device on a solid slope, according to some embodiments of the present invention;

FIG. 6 is a schematic illustration of a water reservoir having a water surface covered by a plurality of floating devices, according to some embodiments of the present invention;

FIG. 7A is a schematic illustration of a single floating device connected to a wire;

FIG. 7B is a schematic illustration of a water surface partially covered by floating devices, wherein the covered area is enclosed by a loop of connected devices, according to some embodiments of the present invention;

FIGS. 8A and 8B show results of experiments performed according to some embodiments of the present invention to investigate the effect of the size of an upper opening of the floating device on the evaporation and temperature;

FIGS. 9A and 9B show results of experiments performed according to some embodiments of the present invention to investigate effect of the inner diameter of a flouting member on the evaporation and temperature;

FIG. 10 shows results of experiments performed according to some embodiments of the present invention to investigate the effect of the percentage of coverage on the evaporation;

FIG. 11 is a schematic illustration of a floating device, in embodiments of the present invention in which the device induces a climate conditioning effect; and

FIG. 12 shows results of experiments performed to study a climate conditioning effect induced by a floating device according to some embodiments of the present invention.

### DESCRIPTION OF SPECIFIC EMBODIMENTS OF THE INVENTION

The present invention, in some embodiments thereof, relates to algae and fish, more particularly, but not exclusively, to a method and floating device for growing fish and/or algae.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present inventor found that while conventional floating devices that are deposited on the surface of the liquid arrange themselves into a cover, they do not promote Algae growth. Algae in reservoirs are conceived as a problem because of possible risk of clogging pipes and releasing toxins. The present inventor found that algae are beneficial to water quality since they produce oxygen, combine some heavy metals and dissolve some harmful substances. Algae that are particularly beneficial are small size algae (e.g., algae having a diameter from 1 mm to 10 mm, or from 1 mm to 5 mm) or micro-size algae (e.g., algae having a diameter less than 1 mm or less than 100 μm). Larger algae are typically less beneficial and are oftentimes undesired.

Conditions that allow algae growth, include light, preferably at wavelength range from about 550 nm to about 850 nm, flow, and nutrients. The present inventor devised a technique which provides conditions that allow growth of small size and micro size algae, and optionally prevent or reduce growth of plankton and/or big leaves algae.

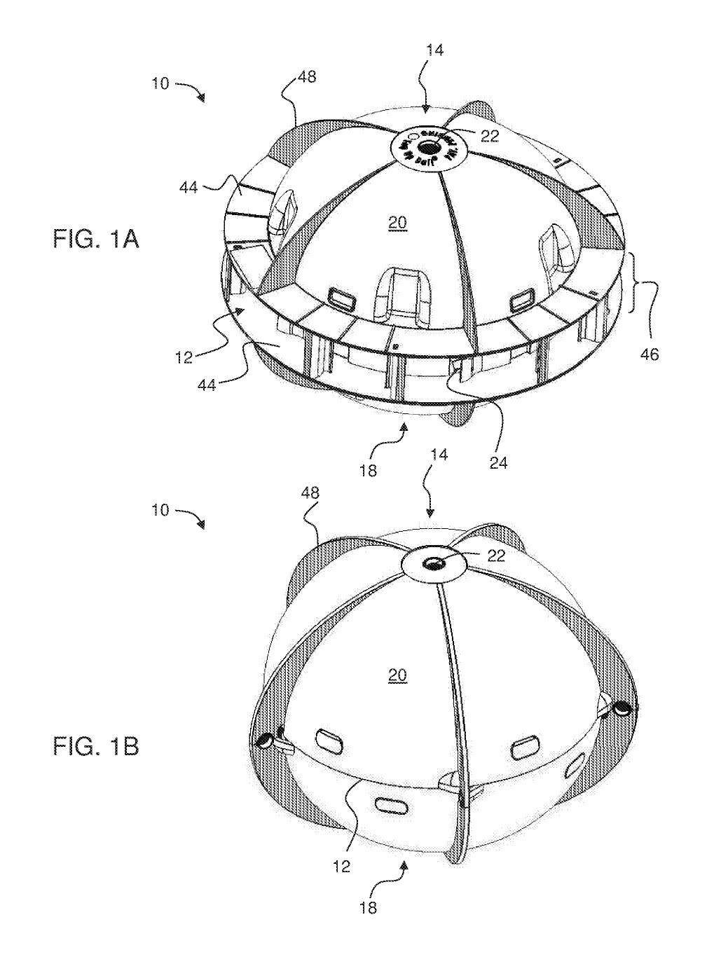

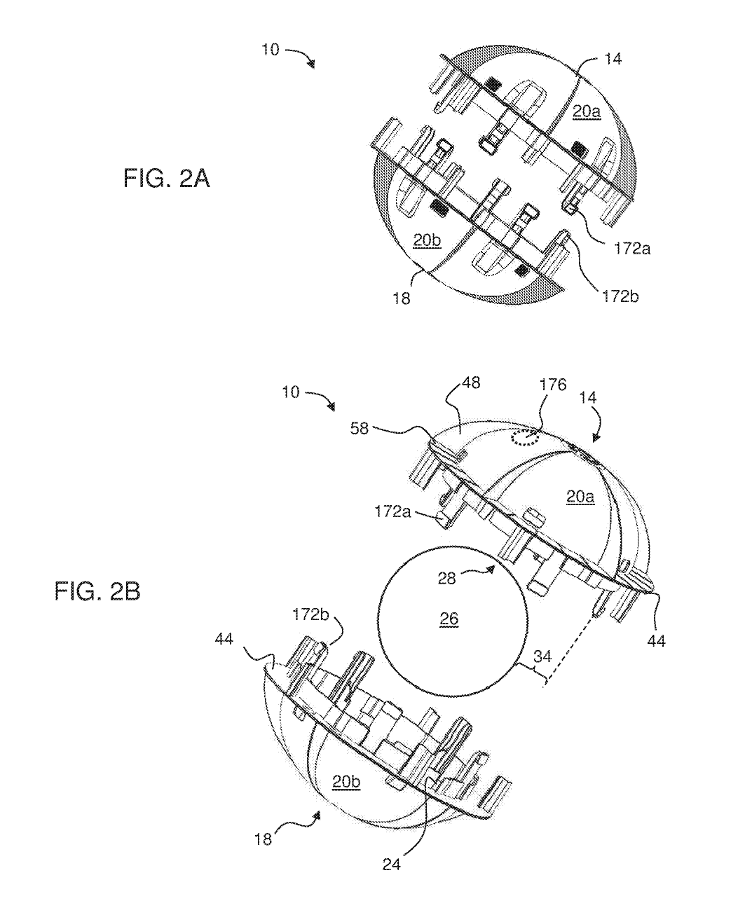

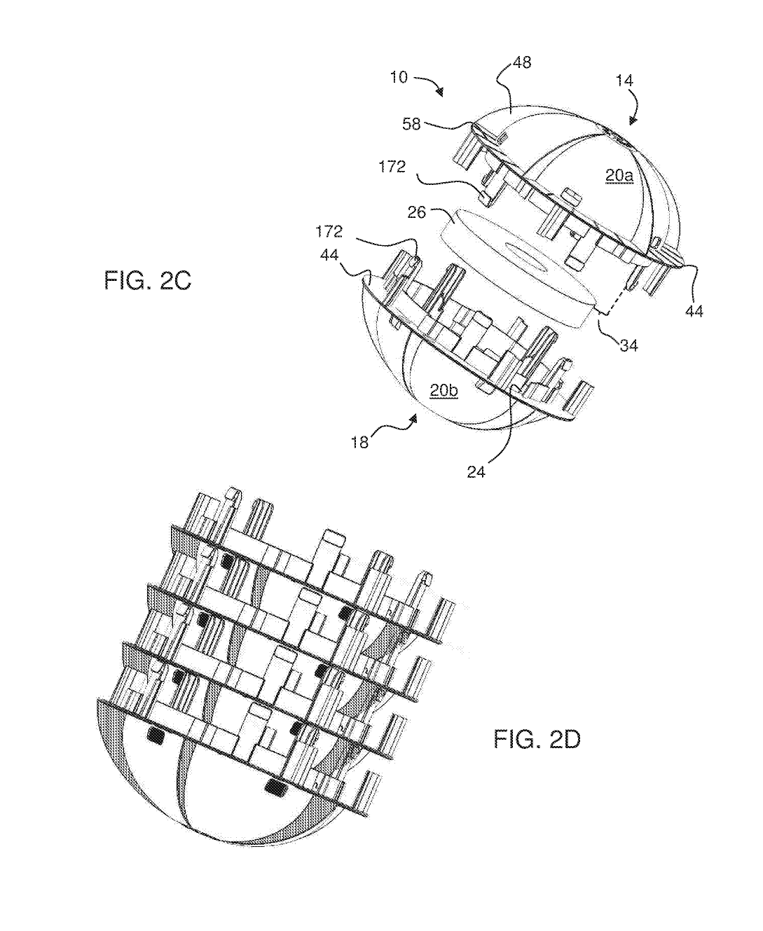

FIGS. 1A and 1B are schematic perspective views and FIGS. 2A-D are schematic exploded views of a floating device 10, according to various exemplary embodiments of the present invention.

Device 10 can be used in many applications. Generally, device 10 can be used in any application in which it is desired to place a physical object on a liquid surface. For example, in some embodiments device 10 is used for growing algae. These embodiments are particularly useful when one or more units of device 10 are placed on the water surface of an aquaculture pond, thereby allowing the fish to be fed by algae growing inside the device. Thus, device 10 can be used as a fish aggregation device. This aggregation of fish, near an exit pipe for example, limits the amount of floating Algae where the risk of clogging pipes is high. In some embodiments device 10 is used for covering a liquid surface, e.g., to reduce evaporation of liquid or any other transfer of thermal energy between the liquid and the environment. In some embodiments device 10 is used for covering, at least partially, an aquaculture pond so as to reduce bird predation. In some embodiments device 10 is used for trapping insects flying at a water surface, wherein the insects enter device 10, but fail to escape once entered.

In some embodiments device 10 is used to grow larva or fish, either newborn fish or adult fish. These embodiments are particularly useful for protecting the larva or fish from predation by other fish.

Device 10 comprises a generally spherical hollow body 20 defined by an equator line 14, a top pole 16 and a bottom pole 18. Body 20 is formed with at least one opening 22 at each of top pole 16 and bottom pole 18, and with an arrangement of openings 24 circumferentially distributed above and below equator line 12. The openings 22 at the top pole preferably have a diameter selected to reduce evaporation of water out of the body. A typical diameter of opening 22 is, without limitation, from about 8 mm to about 12 mm Larger diameters are also contemplated, typically, but not necessarily, in embodiments in which it is desired to let fish enter the device through opening 22. In these embodiments, the diameter of opening 22 can be from about 2 cm to about 4 cm for small-size fish, and from about 4 cm to about 15 cm for larger-size fish.

Device body 20 can be made from two hemispheres 20a and 20b, as illustrated for example, in FIG. 2A. In some embodiments of the present invention hemispheres 20a and 20b are identical in shape, size and optionally also weight. This allows nesting of a plurality of disassembled hemispheres and provides for efficient packing as illustrated in FIG. 2D. The connectors 172 between hemispheres 20a and 20b can be of a snap type to allow quick assembling of the device. In some embodiments, each hemisphere of body 20 is provided with a peripheral rim 44 near the equator line 12, such that when the two hemispheres are assembled, a circular niche 46 is formed between the rims, wherein the rims are below and above the equator line. A configuration with rims 44 is useful when a plurality of floating devices cover a water surface, since it reduces piling of the devices. However, it is not necessary for device 10 to include rims 44, as illustrated in FIG. 1B. A configuration without rims is useful, particularly, but not exclusively, when a single device is employed (e.g., on the surface of an indoor aquarium) or when several spaced apart devices are employed. When rims 44 are employed, openings 24 are preferably formed within the niche 46.

Device 10 optionally and preferably comprises a floating member 26 (see FIGS. 2B-C) inside hollow body 20. Floating member 26 can be made, for example, from a foamed material or any other material with sufficiently low specific weight to allow it to float on the water surface. The largest dimension of the floating member is preferably smaller than the radius of hollow body 20 by at least 5%.

FIG. 2B illustrate an embodiments of the invention in which floating member 26 has an apex 28 constituted to support top pole 14 when floating device 10 is placed on the water surface. For example, floating member 26 can be generally shaped as a sphere, a spheroid (e.g., oblate spheroid or prolate spheroid), an ovoid or the like. The dimensions of floating member 26 are optionally and preferably selected such that a fraction X of its volume submerges under water surface while apex 28 supports top pole 14 to maintain equator line 12 generally at the level of the water surface. Fraction X can be from about 0.03 to about 0.12, or from about 0.05 to about 0.1 or from about 0.06 to about 0.8.

A generally spherical shape for the device has the advantage that the water area for evaporation inside the device is relatively large, thereby enhancing the cooling effect of the device. An additional advantage is that the device withstands high winds since water vapor can escape from the device through the top pole. In some embodiments of the present invention floating member 26 is made of the same material as the body of device 10. This is advantageous from the standpoint of recycling, since both the body of the device and the floating member can be recycled together.

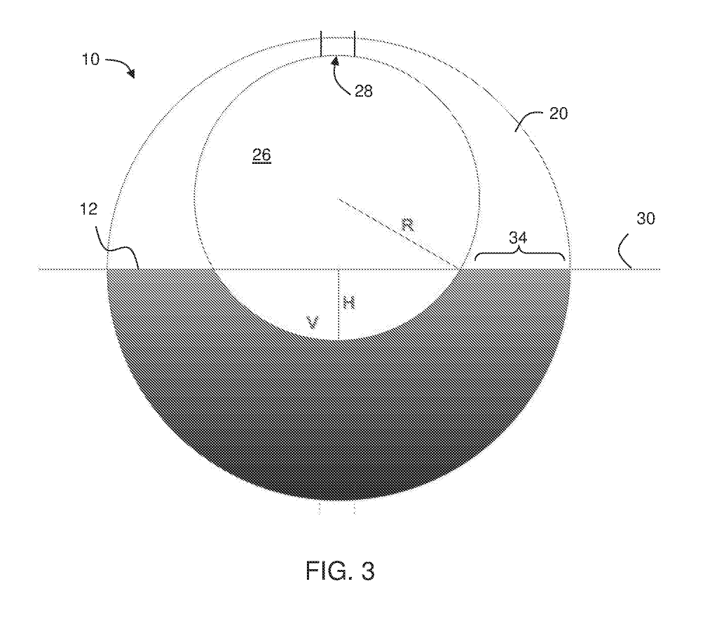

FIG. 3 is a cross-sectional illustration of device 10, once placed on a water surface 30, in embodiments of the invention in which a spherical floating member 26 is employed. As shown, apex 28 supports pole 14 and equator line 12 is generally at the level of water surface 30. In FIG. 3, the radius of floating member 26 is denoted R and the height of the submerged section of floating member 26 is denoted H. The volume V of the submerged section of floating member 26 is V=(1/3)πH²(3R−H), and the total volume V_{T} of floating member 26 is V_{T}=(4/3)πR³. Thus, the fraction X is given by H²(3R−H)/(4R³). For example, when H equals R/3, X equals 2/27. The radius R can be selected based on the water density ρ and the mass M of device 10 without member 26 (approximately the mass of body 20). Specifically, R can be calculated from the equation pV=M. For example, when H equals R/3, ρ equals 1 gr/cm³ and M equals 500 gr, a value of about 11.7 cm is obtained for R.

In general, smaller sizes for member 26 correspond to lower stability of device 10. In various exemplary embodiments of the invention device 10 is made with reduced stability near the tipping point as to enhance the ability of the device to rotate when a bird tries to stand on it. This prevents birds from predating fish. In some embodiments, nails or other tapered elongated structures can be formed on the surface of the device. This is particularly useful, for example, for ponds or reservoirs that are near airports runways, since birds can cause hazard for aircrafts.

In some embodiments of the present invention the body of device 10 has a reduced UV emission, so as to disguise the water beneath device 10.

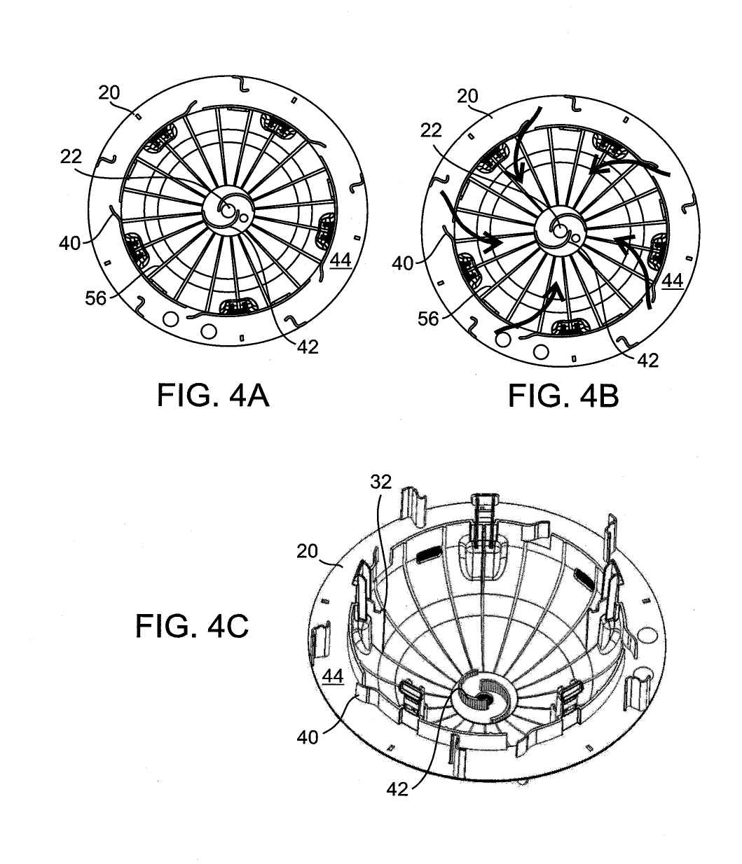

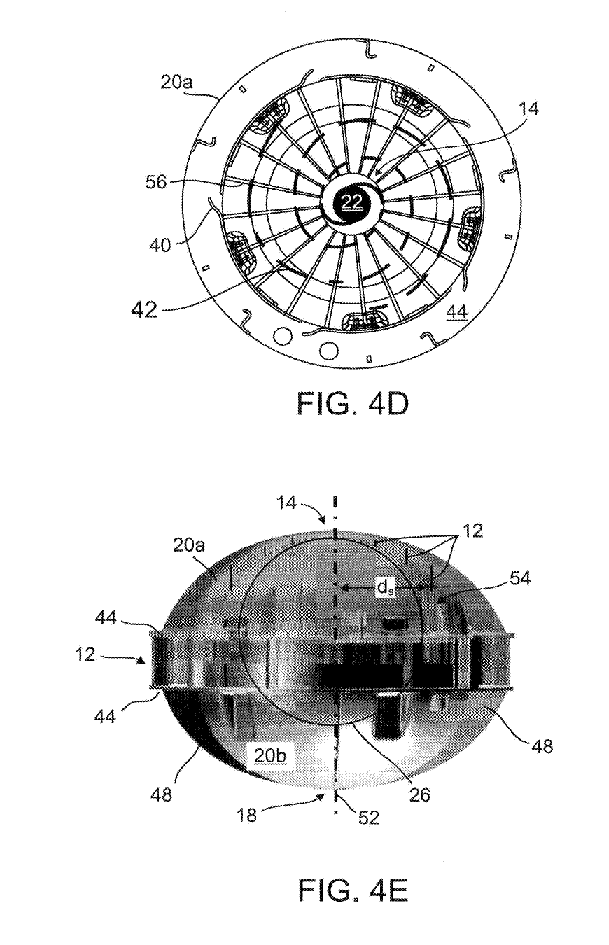

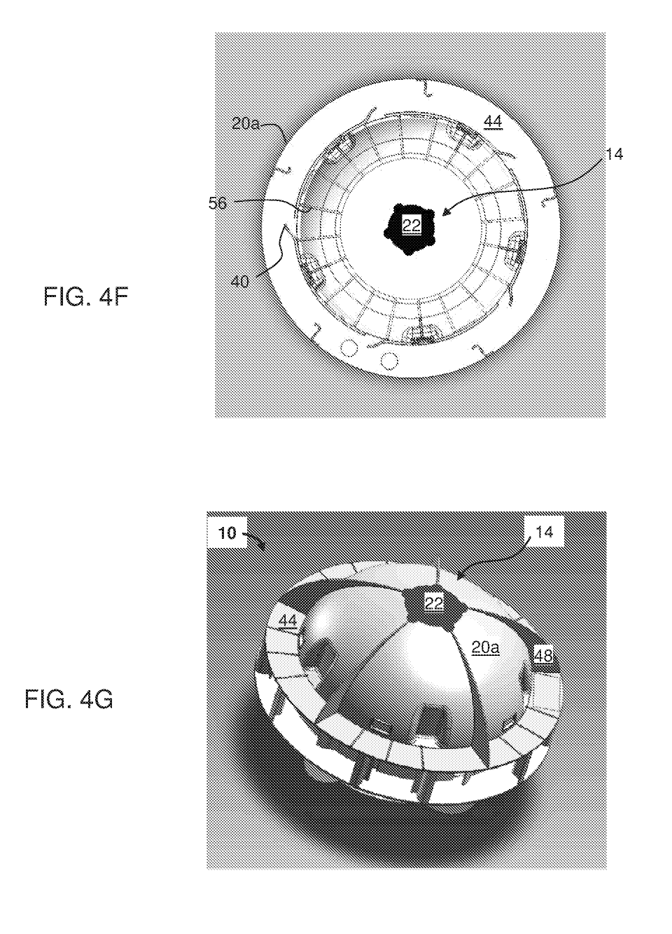

FIG. 2C illustrates an embodiments of the invention in which floating member 26 is generally shaped as a ring. In various exemplary embodiments of the invention the ring is approximately at or near equator line 12 of body 20. In embodiments in which floating member 26 has a flat shape (e.g., a ring) member 26 can be maintained in its position at or near equator line 12 by means of stopper elements 32 (not shown, see FIG. 4C). The inner diameter of the ring is optionally and preferably selected to reduce evaporation and provide for more Algae area growth. Typical value for the inner diameter of the ring is from about 2 cm to about 3 cm. Other diameters are not excluded from the scope of the present invention.

In various exemplary embodiments of the invention there is a gap 34 between the periphery of floating member 26 and the internal wall of device body 20. Gap 34 can be formed by providing member 26 with a diameter that is slightly smaller than the largest diameter of device body 20, or by generating small shape mismatches (e.g., recesses and the like) between the periphery line of member 26 and the equator line 12 of device body 20. The advantage of having gap 34 is that it allows some fluid communication between the top and bottom half of device 10 and facilitates an evaporation process inside the device.

In some embodiments, the openings that are near the equator line (opening 22) are arranged at both sides of floating member 26. Preferably, connectors 172a and 172b are arranged such that when the two hemispheres are assembled, the openings of one hemisphere are azimuthally shifted with respect to the openings of the other hemisphere (for example, a shift of half the distance between adjacent openings). The advantage of this embodiment is that when device 10 is placed on the surface of the water, there is a high likelihood that liquid will enter only to one side of the device through the opening or openings that first contacts the liquid. The influx of liquid into one side of device 10 makes the respective side heavier and provides device 10 with a self-righting property. Thus, the heavier hemisphere is gradually filled with water and the device is oriented such that the openings of the heavier hemisphere are below the water surface. The openings of the other hemisphere remain above the water surface and can serve as venting holes.

FIGS. 4A and 4B illustrate an embodiment in which the inner body 20 comprises a plurality of peripheral flow redirectors 40 arranged at or near the equator line of body 20. For clarity of presentation, FIGS. 4A and 4B only show redirectors 40 arranged at one hemisphere of body 20, but it is to be understood that both hemispheres can include redirectors 40. Redirectors 40 are typically positioned near openings 24 (not shown in FIGS. 4A and 4B). For the hemisphere that is submerged, redirectors 40 serve for redirecting flow of incoming water in a manner that generates circulation of water within the hemisphere. For the hemisphere that remains above the surface of the water redirectors 40 also serve for redirecting incoming air in a manner that generates circulation of air within the hemisphere. The redirected flow of water or air is illustrated as thick arrows in FIG. 4B.

In various exemplary embodiments of the invention device 10 comprises flow redirectors also at the poles of body 20. These flow redirectors are shown at 42, and are positioned to at least partially surround the openings 22 at the poles at the interior of body 20. Each flow redirector 40 is optionally and preferably configured for establishing spiral flow about the respective pole. This is particularly advantageous for bottom pole when it is submerged since it facilitates exit of water through the opening 22 at the pole. Thus, device 10 provides for generally continuous influx of water through the peripheral openings 24 and corresponding efflux of water through the opening at the bottom pole. For the top pole, flow redirector 40 preferably reduces the amount of direct sunlight that enters the hemisphere above the liquid surface.

Flow redirector 42 can comprise a plurality of curved structures separated from each other by at least top pole. A representative example is a curved structure is a cylindrical segment which is a cut from a cylinder by a plane oriented parallel to the cylinder's axis of symmetry. Two curved structure can be positioned at opposite sides of the opening with their concave sides facing each other, but at an offset with respect to each other, thus establishing spiral flow. Flow redirector 42 prevents the floating member 26 from sealing the opening at the upper pole.

When device 10 is used for growing algae, live algae, which have floating ability due to produced bubbles of oxygen, generally occupy the parts of the submerged hemisphere which are close to the water surface for light, wherein dead algae, which lack the floating ability occupy the lower part of the submerged hemisphere. An advantage of flow redirector 42 is that it facilitates the removal of dead algae and other sediments from the device through the opening 22 at the bottom pole.

FIGS. 4D and 4E schematically illustrate embodiments of the invention in which one or more of flow redirectors 42 spirally winds about one of the poles (pole 14 is illustrated in FIGS. 4D and 4E) over the inner surface of the hemisphere (hemisphere 20a is illustrated in FIGS. 4D and 4E). FIG. 4D illustrates hemisphere 20a as viewed from the equator plane, and FIG. 4E illustrates a side view of device 10 in which the hemisphere 20a is illustrated as transparent.

The spiral redirector 42 extends towards equator line 12 so that the distance dₛ from an imaginary axis 52 connecting the two poles 14, 18 to the end of spiral redirector 42 that is farthest from the pole is at least one third or at least one half or at least two thirds of the diameter of body 20. These embodiments are particularly useful in embodiments in which the openings 22 at the pole(s) are relatively large, for example, when device 10 is used for growing fish.

The spiral redirector 42 of the present embodiments serves as a separation member that maintains a gap between the top pole (e.g., pole 14) and the floating member 26, so as not to seal the opening at the top pole and to allow airflow therethrough. Use of non-spiral separation members is also contemplated. The advantage of maintaining the gap between top pole and the floating member is that the airflow through the opening at the pole generates a cooling effect as described herein.

In some embodiments of the present invention the height of spiral redirector(s) 42 monotonically increases away from the pole and toward the equator line 12. The rate of height increment is optionally and preferably selected such that the free end of each spiral redirector 42 (the end that is not connected to body 20 of device 10) engages a hemisphere as shown by the dotted line 54 of FIG. 4E. Hemisphere 54 marks the minimal distance that is allowed between member 26 and body 20, during their relative motion with respect to each other. Optionally and preferably the spiral redirector is segmented in a non-continuous manner. The advantage of this embodiment is that it generates an airflow vector that has non-zero component along redirector 42 and a non-zero component perpendicular to redirector 42.

FIGS. 4F and 4G schematically illustrate embodiments of the invention in which the shape of one or more of the openings 22 at the poles of device 10 are differs from the shape of floating member 26 (not shown in FIGS. 4F and 4G, see, e.g., FIG. 2B) such that when floating member 26 engages the opening 22 at the top pole, sealing of opening 22 by floating member 26 is prevented. These embodiments are also useful when the openings 22 at the pole(s) are relatively large, for example, when device 10 is used for growing fish. The advantage of preventing the sealing of the opening at the top pole is that the airflow through the opening generates a cooling effect as described herein.

FIG. 4F illustrates hemisphere 20a as viewed from the equator plane, and FIG. 4F illustrates a perspective view of device 10. In these embodiments, device 10 is optionally and preferably devoid of any separation member such as a spiral flow redirector, since the shape mismatch between floating member 26 and opening 22 ensures exchange of air between the interior of hemisphere 20a and the environment at all times. In the present embodiments, opening 22 can have shape that mismatches the cross-sectional shape of floating member 26. For example, when floating member has a circular cross-section (e.g., when floating member is spherical), the shape of opening 22 is non-circular. In the representative illustrations shown in FIGS. 4F and 4G, which is not to be considered as limiting, opening 22 has a complex shape formed by a central circle and a plurality of semicircles circumferentially arranged at the periphery of the central circle. Thus, when floating member 26 engages the central circle, there is no sealing of opening 22 since airflow is allowed through the peripheral semicircles.

In some embodiments of the present invention the inner wall of device 10 has roughness features 56 thereon. The roughness features 56 facilitate better attachment of a bio-film to the internal wall of device body 20. These embodiments are particularly useful when device 10 is used for growing algae. Roughness features 56 are optionally and preferably elongated and are oriented generally along meridians of body 20.

Roughness features can additionally or alternatively be formed on peripheral rims 44. These roughness features are shown at 58. Roughness features 58 facilitate growth of bio-film and algae also on rim 44. These embodiments are particularly useful for feeding fish. Roughness features 58 are preferably, but not necessarily, at a side of the peripheral rim which is opposite to the equator line. Roughness features 58 can also be elongated and are preferably oriented along the radial direction of rim 44.

In some embodiments of the present invention device 10 comprises a plurality of external wings 48 distributed on the external of body 20. External wings 48 are optionally and preferably oriented generally along meridians of body 20 (e.g., generally at right angle to equator line 12. Wings 48 allow winds to somewhat rotate the device and avoid piling and overleaping of adjacent devices. The wings also assist in rolling the device back to the water level if reservoir's water level drops. The number of external wings is preferably, but not necessarily, odd (for example, 5 or 7 wings), so as to facilitate rotations of the device by wind forces.

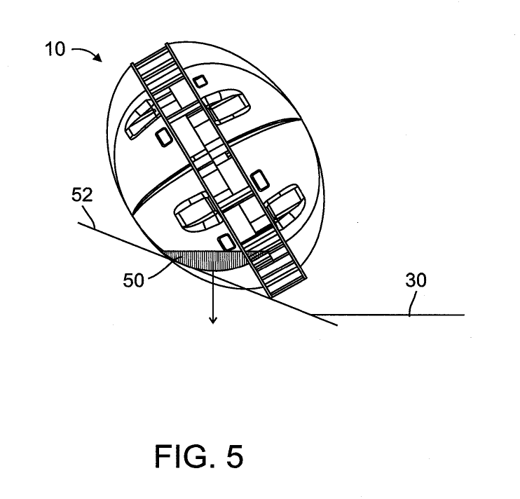

In some embodiments of the present invention the orientation, the shape and locations of wings 48 on body 20 are selected such that the all edges of wings 48 engage the same (imaginary) spherical surface. The advantage of this embodiment is that it does not prevent rolling of device 10 when it arrives to a slope, for example, when the level of liquid is decreased. FIG. 5 is a schematic illustration of device 10 on a solid slope, such as the periphery of a water reservoir and the like. In the illustrated scenario, device 10 already contains an amount of water 50 therein. This can occur following a reduction in the water level in the reservoir. The water in the device effects inclination of the device, and the device begin to roll in the direction of the water level 30.

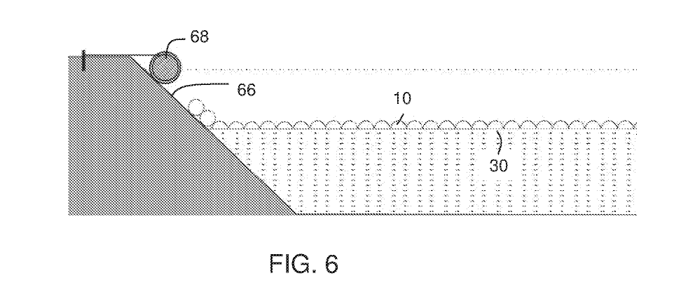

FIG. 6 is a schematic illustration of a water reservoir having a water surface 30 covered by a plurality of floating devices 10. The periphery of the water reservoir has a slope 66 and several floating devices remain on the slope without contacting the water. This situation occurs when the water level is reduced or when there is an excess number of floating devices. In some embodiments of the present invention a barrier 68 is placed at the upper part of slope 66 so as to prevent devices 10 from escaping the reservoir, e.g., by the wind.

Device 10 can be used for covering a water surface of a water reservoir. It is oftentimes desired to cover a water surface so as to limit its exposure to the environment. For example, in aquaculture, certain fish species require a certain temperature range in order to live and grow. Reduction of evaporation from aquaculture ponds with, e.g., water, also reduces salinity of water. Evaporation is also problematic in regions permanently subjected to arid weather or areas temporarily experiencing unusual drought conditions. The reduction of evaporation or the control of energy transfer may be critical to the viability of industry in these geographical regions.

It was found by the present inventor that when device 10 is placed on the surface of liquid such as water, it can be utilized for facilitating vapor condensation. In these embodiments, the color of the upper hemisphere of device 10 is bright so as to reflect broad spectrum of the solar radiation. For example, the upper hemisphere can be white or it can be made of or coated with a sunlight-reflective material. In preferred embodiments in which the two hemispheres are identical in structure as well as in color, both hemispheres have a bright color or are coated with a sunlight-reflective material.

The condensation process, according to some embodiments of the present invention will now be described. During high solar intensity, e.g., from some time (e.g., 1-4 hours) before the noon intensity peak to some time (e.g., 3-4 hours) before sunset, the evaporation of water is significant, and the air above the water surface becomes humid. The humidity is highest near water surface where evaporation takes place. Therefore, the air that enters device 10 through the openings 24 above the water surface is hot and humid. Due to the reflectivity of the water surface, the tendency of hot air to rise upwards, and side winds (when present), most of the air enters through the openings near or at the equator line of device 10. The hot and humid air begins to rise within the upper hemisphere, optionally via a turbulent flow generated, at least in part, by the peripheral flow redirectors 40, until the air interacts with the internal wall of the upper hemisphere.

The upper hemisphere typically contains saturated air. Since this hemisphere reflects broad spectrum of the solar radiation, the temperature of the internal wall is lower than the temperature of the air that have just entered the hemisphere. Thus, as the air reaches the internal wall, condensation occurs, water drops are formed on the internal wall, and air within the anterior of the upper hemisphere becomes dryer. The dryer air exits through opening 22 at the top pole of the hemisphere. Under the effect of gravity, at least some of the condensed water drops slide on the internal wall toward the equator line of device 10, bypass floating member 26 through gap 34, and mix with the water already filling the anterior of the lower hemisphere. Floating member 26 maintains a generally constant volume of water within device body 20, such that excess water exits through the openings 24 that are below the water level.

It was unexpectedly found by the inventor of the present invention that device 10 remains cooler than the environmental air for many hours. The gap between the floating member and the body of the device allows evaporation within the device. Consequently, air is released through the opening at the top pole 14, and device 10 functions essentially as a cooling tower. This allows device 10 to condensate also when the solar intensity is low, for example, early at the morning, and even at night times. Specifically, due to the low temperature of device 10 dew drops are formed on the external wall of the device. The dew drops slide on the external wall and enter the water underneath the device. Since the latent heat of these drops is higher than surrounding water, the water temperature inside the lower hemisphere is higher than that of the surrounding water. It was found by the present inventor that this effect enhances the growth rate of algae.

The low temperature of device 10 also reduces the temperature of the water underneath the device. Thus, when a plurality of devices like device 10 is placed on a surface of a water reservoir, the temperatures of the water surface, hence also the water bulk is reduced. This is an advantage since it increases the quality of water by allowing more dissolved air in. The evaporation-condensation cycle within the device is also advantageous since the condensed water that returns into the reservoir is pure water thus enhancing water quality.

In applications in which evaporation of the body of water is desired (e.g., when device 10 is used for covering an evaporation ponds or part thereof), the upper hemisphere of device 10 can be made dark so as to absorb sunlight. In these embodiments, the temperature of device 10 is higher than the temperature of the liquid on which the device is placed. As a result, the temperature of the liquid at the liquid surface near the device is increased, and the evaporation is higher.

When it is desired to cover a liquid surface for reducing evaporation of liquid or transfer of thermal energy between the liquid and the environment, device 10 can be made reflective to light to reduce the amount of heat that is delivered by radiation. This can be useful both for aquaculture pond and for other types of liquid reservoir. A thermo-chromatic color paint may be used to reflect heat on high temperatures (e.g., white color, silver color, etc) or absorb heat on cold weather (e.g., black, dark blue, etc.). Such paint can be triggered say at 16° C. to change color from silver or white to black. The formation of devices works also as a wind breaker further reducing evaporation. The devices can also include fluorescent material on their external surface increasing oxygen producing time by Algae.

In some embodiments of the present invention the body 20 of device 10 is generally transparent to visible light, and floating member 26 has a dark color. In some embodiments of the present invention body 20 is transparent also to light in at least one range selected from the group consisting of infrared light (e.g., near infrared light) and ultraviolet light.

As used herein “transparent” include any of completely transparent, semi transparent and partly transparent. For example, “transparent body” may relate to a body of device 10 which allows at least 20% or at least 40% or at least 60% or at least 80% or at least 90% of incident light at any wavelength within the respective spectrum (visible, infrared, ultraviolet) to pass through the walls of the body into the interior of the body.

As used herein, “dark color” refers to a color which is either black or approaching black in hue, including, for example, dark grey, dark blue, dark green, dark brown, and the like. As used herein, “black” refers to “optically black.” As used herein “optically black” refers to a material which appears black and opaque on visual inspection. In certain embodiments, floating member 26 has is optically black.

The darkness of floating member 26 can be quantified using the CIE 1976 (L*, a*, b*) color space (also known as the CIELAB). For any color over the CIELAB, the L* coordinate can be viewed as the lightness of the color, wherein L*=0 corresponds to black and L*=100 corresponds to diffuse white. In various exemplary embodiments of the invention floating member 26 has a color characterized by a coordinate L* over the CIELAB, wherein L* is less than 50 or less than 40 or less than 30 or less than 20 or less than 10 or less than 5, e.g., L*=0.

The L* value in CIE1976 color space can be measured, for example, using a spectrocolorimeter, such as, but not limited to, the BLACK-Comet CXR SpectroColorimeter marketed by StellarNet, Inc., Florida, USA.

The present inventor has unexpectedly discovered that a floating device according to some embodiments of the present invention can generate a climate conditioning effect. Specifically, it was found that at low temperature, a floating device having a dark floating member and optionally also a transparent body can use solar energy to increase the temperature of water in a water reservoir, wherein at higher temperatures such a floating device can reduce the temperature of the water in the water reservoir. This property of the floating device is particularly useful for aquaculture ponds, for example, for the purpose of reducing fish mortality and increasing yield.

Without wishing to be bound by any particular theory, the following explanation by the present inventor can aid the understanding of the unexpected climate conditioning effect. FIG. 11 shows device 10 in embodiments in which body 20 is transparent (shown as a double line) and member 26 is spherical and dark (shown hatched). The interior of the upper hemisphere 20a of body 20 is filled with air and the interior of the lower hemisphere 20b of body 20 is filled with water. In outdoor locations that receive solar energy (typically, but not necessarily, direct sunlight) two conflicting effects co-exist in the interior of body 20. A heating effect caused by virtue of the darkness of floating member 26, and a cooling effect caused by virtue of water evaporation and airflow through the opening at the upper pole as further detailed hereinabove. Thus, the thermodynamic condition in the interior of device 10 is governed by a detailed balance equation wherein when the temperature of the water is high, there is more evaporation and the dominant effect is cooling, and when the temperature of the water is low, there is less evaporation, and the dominant effect is heating.

The extent of the two effects is balanced at a balance temperature which depends on the shape, color, and dimensions of device 10 and the openings in body 20. In experiments performed by the present inventors (see Example 3) with a specific prototype device, a balance temperature of about 16° C. It is appreciated that other dimensions and colors can provide other values for the balance temperature.

The dark color of member 26 can be only over the external surface of member 26, in which case member 26 can be made from a non-dark material and be coated by a dark layer. Alternatively, member 26 can be made of a dark-color material, in which case both the bulk and the external surface of member 26 are dark. In some embodiments of the present invention member 26 has a sufficiently high thermal conductivity (e.g., at least 4 W/(m·° K)). The advantage of these embodiments is that the coating layer can facilitate transfer of heat from floating member 26 to the water.

A sufficiently high thermal conductivity can be ensured by coating member 26 by a coating layer made of a thermally conductive material. A sufficiently high thermal conductivity can also be ensured by incorporating a thermally conductive material into the bulk of member 26, e.g., during the fabrication process (e.g., injection molding) of member 26.

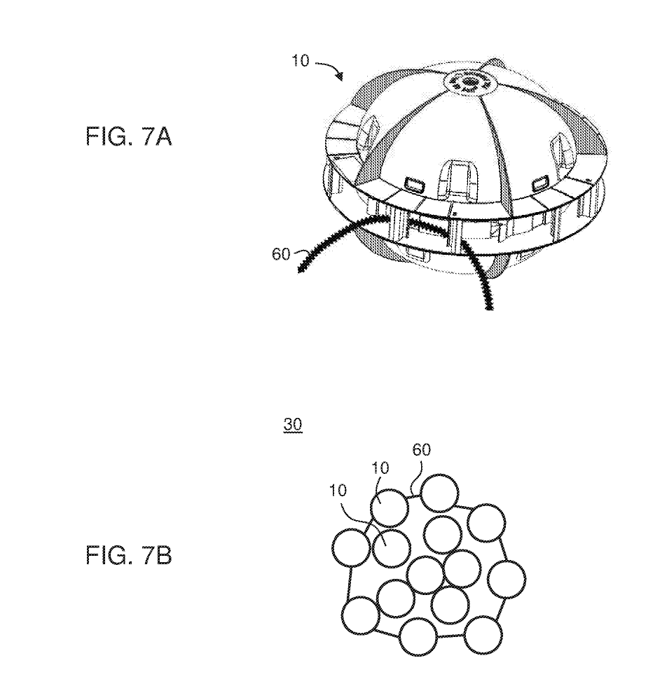

When it is desired to cover a partial area of the water surface, several devices similar to device 10 can be connected, for example, by a synthetic wire, to form a loop of connected floating devices on the water surface, and other floating devices, which can also be similar to device 10 can be placed on the water surface in the area defined by the connected devices. FIG. 7A shows a single floating device 10 connected to a wire 60, and FIG. 7B shows a water surface 30 partially covered by floating devices, wherein the covered area is enclosed by a loop of connected devices. It was unexpectedly found by the present inventor that it is economically sufficient to cover from about 65% to about 85% of the water surface, wherein any additional coverage provides only marginal benefits. Thus, according to some embodiments of the present invention there is provided a method for reducing evaporation. The method comprises partially covering a water surface by floating devices, such as, but not limited to, device 10, wherein the floating device occupy from about 65% to about 85% or from about 70% to about 80% of the total area of the water surface.

When it is desired to device 10 for trapping insects (e.g., mosquito) flying at a water surface, device 10 is placing on the water surface, the insects are allowed to enter device 10 through openings 22 and/or 24. Optionally, an insect attractor is placed in device 10. However, this is not necessary since the insects are attracted to the interior of device 10 by virtue of the thermodynamics of device 10 and/or the oxygen produced by the algae if present in the device, and the presence of CO₂. It is also not required to include insecticides or the like in device 10 since the trapped insects are extinct by the heat within the device 10 or stick to the wet inner walls. Yet, use of insecticides within device 10 is not excluded from the scope of some embodiments of the present invention.

In some embodiments of the present invention device 10 comprises a light source 176 (see FIG. 2B), positioned at the inner side of device 10, at least in the upper hemisphere. Light source 176 can be for example, a light emitting diode. Light source 176 can include or be connected to a power source, such as, but not limited to, a solar power source that collects energy from solar radiation during day time and provides electrical power during night time. Light source 176 can facilitate increased algae production during night time.

In some embodiments of the present invention device 10 comprises a dissolvable substance, typically at the inner side of the lower hemisphere. The dissolvable substance is preferably in solid form and may be packed in a slow-release structure as known in the art. The substance can be selected to improve water quality.

A representative example of dissolvable substances suitable for the present embodiments includes, but is not limited to, copper sulfate. The flow of water inside the lower hemisphere distributes the substances therein.

Optionally, the body of the device 10 comprises additives selected to selectively allow light of wavelength within a predetermined wavelength range (e.g., from about 550 nm to about 850 nm) to enter into the device, while preventing or reducing entry of light of other wavelengths.

Typical sizes and weights for the floating device of the present embodiments are as follows. The radius of body 20, excluding external wings 48, is from about 3 cm to about 120 cm or from about 3 cm to about 17 cm or from about 17 cm to about 120 cm or from about 20 cm to about 120 cm or from about 50 cm to about 120 cm.

Lower radii (e.g., 3-4 cm) are particularly useful when device 10 is used for feeding fish in an indoor aquarium, and larger radii are useful when device 10 is used in outdoor water reservoir or aquaculture pond. Radii above 17 cm are particularly useful when it is desired to allow fish to enter the device. The mass of device 10 is preferably from about 50 gr to about 5000 gr or from about 50 gr to about 600 gr.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

The performances of a prototype floating device according to some embodiments of the present invention were tested.

The prototype floating device included a generally spherical hollow body, 15 cm in diameter, formed with openings at the poles and above and below the equator line, and a floating separator member at the level of the equator with a gap between separator and body. The separator was a round disk, 6 cm in radius, made of polystyrene.

The prototype device was placed on a water surface within an open water tank having a surface area of about 1200 cm². The depth of the water in the tank was about 40 cm. The temperatures of the prototype device, the environmental air and the water were monitored over a period of 24 hours. Temperature measurements were collected approximately every 60 minutes.

### Example 1

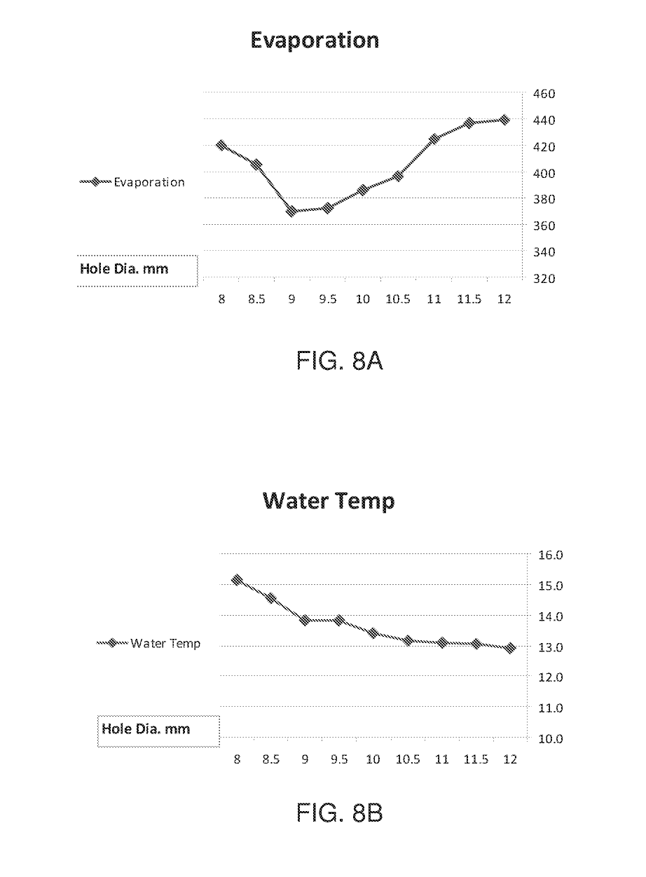

The effect of the size of the upper opening 22 on the evaporation and temperature was tested. Prototype floating device with different sizes (from about 8 mm to about 12 mm) of the upper opening 22 were used in the experiment. The results are provided in Table 1, below, and in FIGS. 8A-B, where FIG. 8A is a graph showing the evaporation (in milliliters) and FIG. 8B is a graph showing the temperature (in degrees Celsius), as a function of the diameter of the opening.

TABLE 1

| | Diameter | | | | | | | | |
|---|---|---|---|---|---|---|---|---|---|
| | 8 | 8.5 | 9 | 9.5 | 10 | 10.5 | 11 | 11.5 | 12 |
| Evaporation 1 | 426 | 420 | 412 | 370 | 383 | 396 | 430 | 427 | 437 |
| Water Temp | 15.6 | 14.9 | 14.4 | 14.3 | 14 | 13.5 | 13.6 | 13.2 | 13.0 |
| Evaporation 2 | 441 | 416 | 391 | 363 | 390 | 387 | 425 | 451 | 451 |
| Water Temp | 14.8 | 14.8 | 14.2 | 14.4 | 14.1 | 14.2 | 13.8 | 13.7 | 13.6 |
| Evaporation 3 | 412 | 439 | 350 | 352 | 375 | 372 | 411 | 427 | 423 |
| Water Temp | 16.1 | 14.5 | 13.8 | 13.7 | 13.3 | 13.1 | 12.7 | 13.2 | 13.1 |
| Evaporation 4 | 432 | 411 | 375 | 377 | 396 | 406 | 396 | 422 | 444 |
| Water Temp | 14.7 | 14.0 | 13.1 | 12.9 | 12.3 | 12.5 | 12.4 | 12.8 | 12.7 |
| Evaporation 5 | 412 | 385 | 340 | 392 | 382 | 425 | 445 | 465 | 458 |
| Water Temp | 14.3 | 14 | 13.1 | 13.3 | 12.9 | 12.5 | 12.8 | 13.1 | 12.9 |
| Evaporation 6 | 396 | 360 | 351 | 381 | 389 | 395 | 443 | 428 | 423 |
| Water Temp | 15.2 | 15.1 | 14.3 | 14.2 | 13.8 | 13.1 | 13.2 | 12.3 | 12.1 |
| Avg Evaporation | 420 | 405 | 370 | 373 | 386 | 397 | 425 | 437 | 439 |
| Avg Temp | 15.1 | 14.6 | 13.8 | 13.8 | 13.4 | 13.2 | 13.1 | 13.1 | 12.9 |
| Hole Diameter | 8 | 8.5 | 9 | 9.5 | 10 | 10.5 | 11 | 11.5 | 12 |

This Example demonstrates that the evaporation reaches a minimum for an opening diameter of approximately 9.5 mm for the given device diameter.

### Example 2

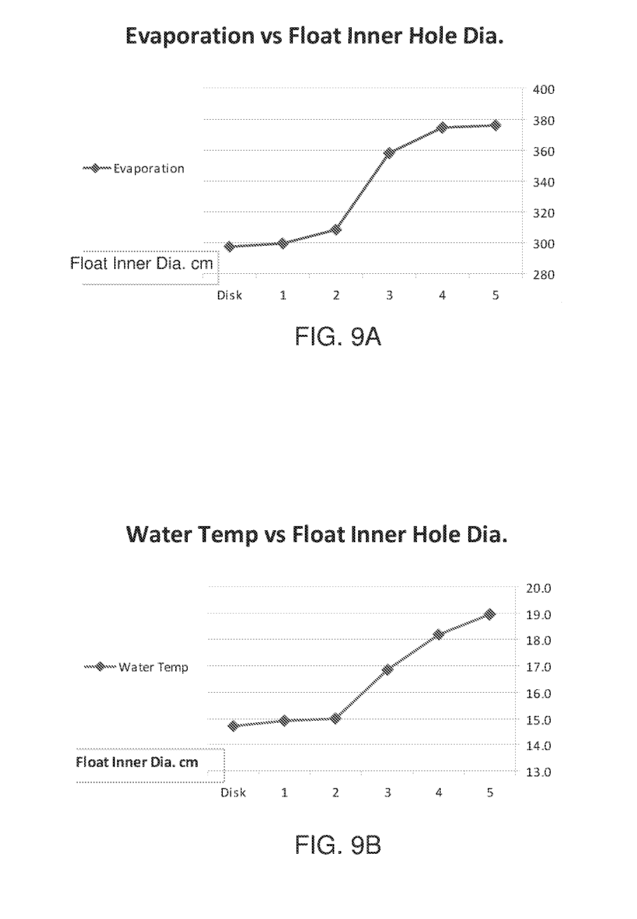

The effect of the inner diameter of a flouting member 26 shaped as a ring on the evaporation and temperature was tested. Prototype floating device with different inner diameters (from 0 cm to about 5 cm) of the flouting member 26 were used in the experiment. The results are provided in Table 2, below, and in FIGS. 9A-B, where FIG. 9A is a graph showing the evaporation (in milliliters) and FIG. 9B is a graph showing the temperature (in degrees Celsius), as a function of the inner diameter of the floating member.

TABLE 2

| | Ring Inner Dia. | | | | | |
|---|---|---|---|---|---|---|
| | 0 (Disk) | 1 | 2 | 3 | 4 | 5 |
| Evaporation 1 | 275 | 280 | 284 | 294 | 303 | 323 |
| Water Temp | 15.0 | 15.1 | 15.6 | 17.3 | 18.6 | 18.3 |
| Evaporation 2 | 363 | 344 | 353 | 407 | 428 | 427 |
| Water Temp | 14.1 | 14.3 | 14.6 | 16.8 | 18.3 | 18.9 |
| Evaporation 3 | 347 | 323 | 335 | 396 | 405 | 397 |
| Water Temp | 15.3 | 15.4 | 15.6 | 17.1 | 18.4 | 18.9 |
| Evaporation 4 | 306 | 323 | 313 | 371 | 401 | 398 |
| Water Temp | 15.2 | 15.3 | 14.8 | 16.8 | 18.6 | 19.6 |
| Evaporation 5 | 194 | 221 | 247 | 380 | 386 | 376 |
| Water Temp | 14.6 | 14.9 | 14.6 | 16.7 | 17.7 | 18.6 |
| Evaporation 6 | 297 | 304 | 318 | 298 | 323 | 333 |
| Water Temp | 14.1 | 14.4 | 14.7 | 16.3 | 17.4 | 19.4 |
| Avg Evaporation | 297 | 299 | 308 | 358 | 374 | 376 |
| Avg Temp | 14.7 | 14.9 | 15.0 | 16.8 | 18.2 | 19.0 |

This Example demonstrates that evaporation and temperature are generally the same for any diameter less than about 2 cm. Thus, for the prototype device used in the present experiment it is not required to reduce the inner diameter to less than 2 cm.

### Example 3

The effect of the percentage of coverage on the evaporation was tested. In this example, the amount of evaporation was measured for an uncovered water surface, a water surface partially covered by the prototype floating device, and a water surface partially covered by non-hollow plastic disks (Bottle caps).

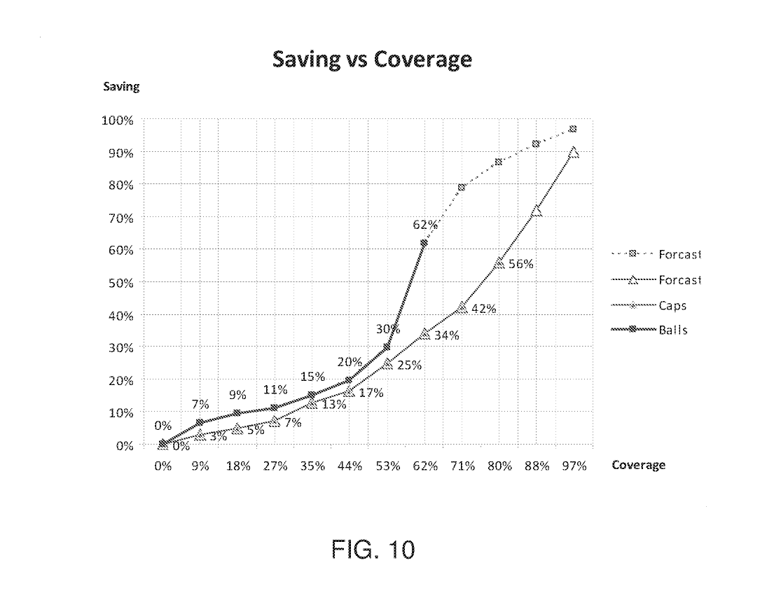

The results are provided in Table 3, below, and in FIG. 10, where FIG. 10 is a graph showing the saving (in percentage) as a function of the percentage of coverage.

The saving in percentage is defined as 100−100*Y/X where X is the amount of evaporation in the control experiment, and Y is the amount of evaporation using the prototype floating device. The curves in FIG. 10 were extrapolated to 100%.

In Table 3, the first column shows the area covered expressed in percentage of to the total water area, the second column shows the number of balls or caps that were used, the third column shows the amount of water added in ml to an uncovered bowl, the fourth column shows the amount of water added to the covered bowl, the fifth column shows the saving in percentage gained by the ball covered bowl, and the sixth column shows the saving in percentage gained by caps covered bowl.

TABLE 3

| Caps | Balls | Test | Control | No. of floating devices | Coverage |
|---|---|---|---|---|---|
| 0% | 0% | 1201 | 1200 | 0 | 0% |
| 3% | 7% | 1235 | 1322 | 1 | 9% |
| 5% | 9% | 1434 | 1582 | 2 | 18% |
| 7% | 11% | 1330 | 1494 | 3 | 27% |
| 13% | 15% | 1152 | 1354 | 4 | 35% |
| 17% | 20% | 1280 | 1591 | 5 | 44% |
| 25% | 30% | 1121 | 1596 | 6 | 53% |
| 34% | 62% | 588 | 1542 | 7 | 62% |
| 42% | 79% | 320 | 1500 | 8 | 71% |
| 56% | 87% | 200 | 1500 | 9 | 80% |
| 72% | 92% | 120 | 1500 | 10 | 88% |
| 90% | 97% | 50 | 1500 | 11 | 97% |

This Example demonstrates that it is economically sufficient to cover from about 65% to about 85% of the water surface, wherein any additional coverage provides only marginal benefits.

### Example 4

The effect of heating and cooling of water covered by the floating device of the present embodiments was tested. In this example, the temperature of water was measured for an uncovered water reservoir, and for a water reservoir partially covered by a prototype floating device. The prototype floating device included a transparent body and a black spherical floating member. The prototype floating device covered about 65% of the reservoir's surface.

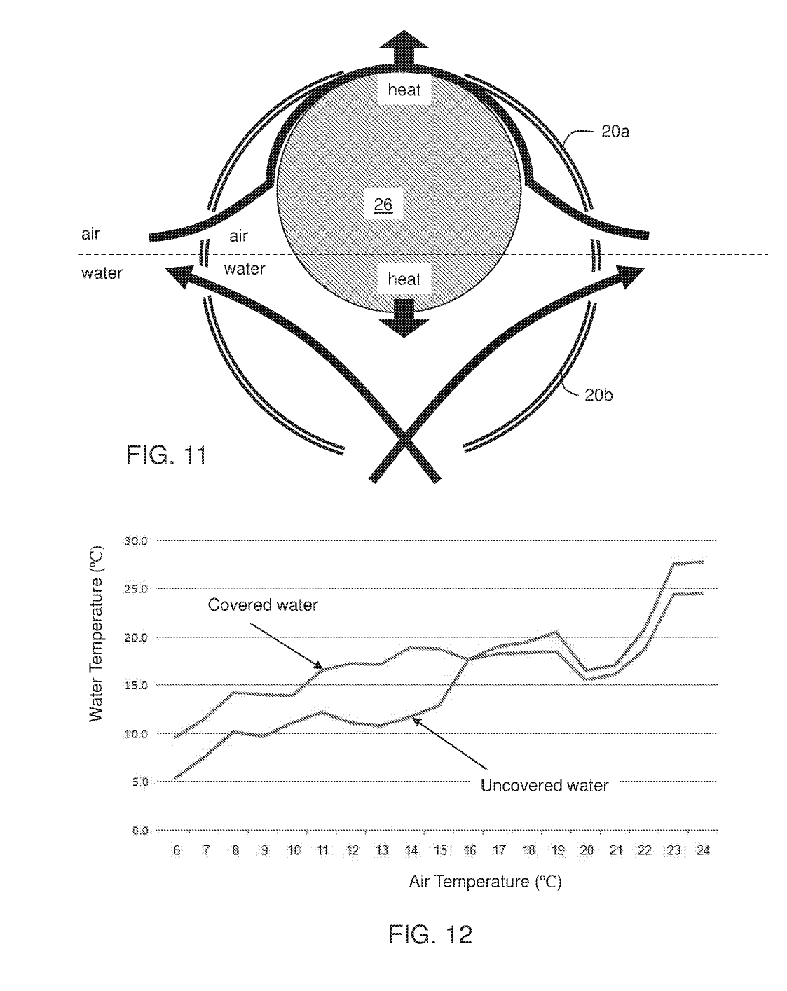

The water temperatures and the ambient air temperature were measured 212 times over a period of eight days, and the average temperature was calculated. The results are shown in FIG. 12, in the form of a graph that describes the water temperature as a function of the ambient air temperatures. As shown, the floating device generates a climate conditioning effect. Specifically, when the ambient air temperature is low (below about 16° C., in the present example), the floating device increases the temperature, and when the ambient air temperature is high (above about 16° C., in the present example), the floating device reduces the temperature.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-59: (canceled)

60. A method of growing algae, comprising placing on a water surface a floating device, and allowing algae culture to grow therein, wherein said floating device comprises:
a generally spherical hollow body defined by an equator line, a top pole and a bottom pole; and
a floating member inside said hollow body; said body being formed with at least one opening at each of said top and said bottom poles, and with an arrangement of openings circumferentially distributed above and below said equator line.

61. A method of growing fish, comprising placing on a water surface a floating device, and allowing fish to enter said floating device, wherein said floating device comprises:
a generally spherical hollow body defined by an equator line, a top pole and a bottom pole, said body being formed with at least one opening at each of said top and said bottom poles, said at least one opening being sizewise compatible with a size of the fish; and
a non-flat floating member, being inside said hollow body and having an apex constituted to support said top pole when the floating device is placed on said water surface, wherein sealing of said opening at said top pole by said floating member is prevented.

62. The method according to claim 60, wherein said floating member is generally shaped as a ring.

63. The method of claim 62, wherein an inner diameter of said ring is selected to reduce evaporation.

64. The method according to claim 60, wherein a largest dimension of said floating member is smaller than a radius of said hollow body by at least 5%.

65. The method according to claim 60, wherein said floating member is generally shaped as a sphere or a spheroid.

66. The method according to claim 60, wherein said floating member has dimensions selected such that a fraction of its volume submerges under said water surface while an apex of said floating member supports said top pole to maintain said equator line generally at a level of said water surface, wherein said fraction is from about 0.03 to about 0.12.

67. The method according to claim 60, wherein said floating device comprises flow redirectors at least partially surrounding each of said openings at said top and said bottom poles at an interior of said body.

68. The method according to claim 60, wherein said opening at said top pole has a diameter selected to reduce evaporation.

69. The method according to claim 60, wherein an inner wall of said body has roughness features thereon.

70. The method according to claim 69, wherein said roughness features are elongated and oriented generally along meridians of said body.

71. The method according to claim 60, wherein said floating device comprises a pair of peripheral rims, mounted or formed on an external wall of said body above and below said equator line and generally parallel thereto.

72. The method according to claim 71, wherein at least one of said peripheral rims is formed with roughness features.

73. The method according to claim 60, wherein said floating device comprises a plurality of external wings, mounted or formed on an external wall of said body generally along meridians thereof.

74. The method according to claim 60, wherein said body is transparent to visible light and said floating member has a dark color.

75. The method according to claim 60, wherein said floating member comprises a thermally conductive material.

76. The method according to claim 60, wherein the device further comprises a light source positioned at an upper hemisphere of said body.

77. The method according to claim 60, wherein the device comprises a dissolvable substance placed at the inner side of a lower hemisphere of the device.

78. The method according to claim 60, wherein said body comprises additives selected to selectively allow transmission of light having wavelength from about 550 nm to about 850 nm.

79. A floating device for use on a water surface, comprising:
a generally spherical hollow body defined by an equator line, a top pole and a bottom pole; and
a non-flat floating member, generally shaped as a sphere or a spheroid, being inside said hollow body and having an apex constituted to support said top pole when the floating device is placed on said water surface;
said body being formed with at least one opening at each of said top and said bottom poles, and with an arrangement of openings circumferentially distributed above and below said equator line.
